(19) United States
(12) Patent Application Publication
(10) Pub. No.: US 2023/0190537 A1
(43) Pub. Date: Jun. 22, 2023

(54) ABSORBENT ARTICLE

(71) Applicant: The Procter & Gamble Company, Cincinnati, OH (US)

(72) Inventors: Wei WU, Beijing (CN); Gueltekin ERDEM, Cincinnati, OH (US); Chunmin CHENG, Cincinnati, OH (US); Koichi MORIMOTO, Beijing (CN); Yi YUAN, Beijing (CN); Meng CHEN, Beijing (CN); Xiuyan TANG, Beijing (CN)

(21) Appl. No.: 18/063,698

(22) Filed: Dec. 9, 2022

(30) Foreign Application Priority Data
Dec. 20, 2021 (WO) PCT/CN2021/139618
Feb. 25, 2022 (WO) PCT/CN2022/077997

(51) Int. Cl. A61F 13/15; A61F 13/537

(52) U.S. Cl. CPC A61F 13/15203; A61F 13/53752; A61F 2013/51452

(57) ABSTRACT

Disclosed is an absorbent article comprising a topsheet, a backsheet, an absorbent core disposed between the topsheet and the backsheet, and an outer cover layer for covering the garment-facing side of the backsheet. The absorbent article has a Thickness Under Compression of from about 2.7 mm to about 4.0 mm. The outer cover layer is formed by a muti-fiber layer nonwoven having a basis weight from about 16gsm to about 35gsm and comprising a garment facing layer and a wearer facing layer. The garment facing layer comprises fibers having a diameter of about 11 µm or less, or from about 7 µm to about 11 µm, and the wearer facing layer comprises fibers having a diameter of about 13 µm or more, or from about 13 µm to about 24 µm. A weight ratio of the garment facing layer is from about 20% to about 70% of the multi-fiber layer nonwoven.

### CROSS REFERENCE TO RELATED APPLICATIONS

This application claims priority under 35 U.S.C. § 119 to Chinese PCT Patent Application Serial No. PCT/CN2021/139618, filed on Dec. 20, 2021 and Chinese PCT Patent Application Serial No. PCT/CN2022/077997, filed on Feb. 25, 2022, the entire disclosures of which are hereby incorporated by reference.

### FIELD

The invention relates to absorbent articles which are particularly thin, flexible, comfortable and having improved softness.

### BACKGROUND

Disposable absorbent articles such as diapers and adult incontinence products are well known in the art. Such disposable articles are designed to absorb and contain body exudates, in particular large quantity of urine. These absorbent articles comprise several layers, for example a topsheet, a backsheet and in-between an absorbent core, among other layers.

One function of an absorbent core is to absorb and retain the bodily exudates for a prolonged amount of time, for example, overnight for a diaper, minimize re-wet to keep the wearer dry, and avoid soiling of clothes or bed sheets. Some currently marketed absorbent articles comprise absorbent cores comprising an absorbent material which is a blend of comminuted wood pulp (i.e., airfelt) with superabsorbent polymers (SAP) in particulate form, or SAP particulate forms encompassed in high loft material. One trade off of absorbent articles which are thin and have a high percentage of SAP particles are a negative touch feeling known as “grainy feel” coming from the SAP particles. Further, by using materials which are advantageous for maintaining the structure of the thin absorbent article may negatively affect the overall flexibility of the absorbent article.

Meanwhile, softness, flexibility and/or cushiony feel are some prioritized sensory requirements for a high quality absorbent article.

Based on the foregoing, there is a need for an absorbent article providing improved softness and flexibility, while maintaining absence of grainy feel and maintain absorbency. There is also a need for providing such an absorbent article which can be economically made.

### SUMMARY

The present invention is directed to an absorbent article comprising a liquid permeable topsheet, a liquid impermeable backsheet, an absorbent core disposed between the topsheet and the backsheet, and an outer cover layer for covering the garment-facing side of the backsheet,
wherein the absorbent article has a Thickness Under Compression of from about 2.7 mm to about 4.0 mm, and
wherein the outer cover layer is formed by a muti-fiber layer nonwoven having a basis weight of from about 16gsm to about 35gsm and comprising a garment facing layer and a wearer facing layer, the garment facing layer comprising fibers having a diameter of about 11 µm or less, preferably from about 7 µm to about 11 µm, and the wearer facing layer comprising fibers having a diameter of about 13 µm or more, preferably from about 13 µm to about 24 µm, wherein the weight ratio of the garment facing layer is from about 20% to about 70% of the multi-fiber layer nonwoven.

### BRIEF DESCRIPTION OF THE DRAWINGS

While the specification concludes with claims particularly pointing out and distinctly claiming the subject matter which is regarded as forming the present invention, it is believed that the invention will be better understood from the following description which is taken in conjunction with the accompanying drawings and which like designations are used to designate substantially identical elements, and in which:

FIG. 1A is a perspective view of one embodiment of an absorbent article of the present invention.

FIG. 1B is a schematic view of one embodiment of an absorbent article of the present invention showing the front side of the article.

FIG. 2A is a schematic plan view of one embodiment of an absorbent article of the present invention with the seams unjoined and in a flat uncontracted condition showing the garment facing surface.

FIG. 2B is schematic plan view of another embodiment of an absorbent article of the present invention showing the wearer facing side.

FIGS. 3A-3C are SEM images of a cross-sectional view of a multi-fiber layer nonwoven of the present invention.

FIG. 4 is a transverse schematic cross section view of an embodiment of the present invention in the crotch region with the thickness (Z direction) exaggerated.

### DEFINITIONS

As used herein, the following terms shall have the meaning specified thereafter:

“Absorbent article” refers to articles of wear which may be in the form of pants, taped diapers, incontinent briefs, feminine hygiene garments, and the like. The “absorbent article” may be so configured to also absorb and contain various exudates such as urine, feces, and menses discharged from the body.

“Pant” refers to disposable absorbent articles having a pre-formed waist and leg openings. A pant may be donned by inserting a wearer’s legs into the leg openings and sliding the pant into position about the wearer’s lower torso. Pants are also commonly referred to as “closed diapers”, “prefastened diapers”, “pull-on diapers”, “training pants” and “diaper-pants”.

“Longitudinal” refers to a direction running substantially perpendicular from a waist edge to an opposing waist edge of the article and generally parallel to the maximum linear dimension of the article.

“Transverse” refers to a direction perpendicular to the longitudinal direction.

“Proximal” and “distal” refer respectively to the position closer or farther relative to the longitudinal center of the article.

“Wearer facing” and “garment-facing” refer respectively to the relative location of an element or a surface of an element or group of elements. “Wearer facing” implies the element or surface is nearer to the wearer during wear than some other element or surface. “Garment-facing” implies the element or surface is more remote from the wearer during wear than some other element or surface (i.e., element or surface is proximate to the wearer’s garments that may be worn over the disposable absorbent article).

“Disposed” refers to an element being located in a particular place or position.

“Joined” refers to configurations whereby an element is directly secured to another element by affixing the element directly to the other element and to configurations whereby an element is indirectly secured to another element by affixing the element to intermediate member(s) which in turn are affixed to the other element.

“Film” refers to a sheet-like material wherein the length and width of the material far exceed the thickness of the material. Typically, films have a thickness of about 0.5 mm or less.

“Water-permeable” and “water-impermeable” refer to the penetrability of materials in the context of the intended usage of disposable absorbent articles. Specifically, the term “water-permeable” refers to a layer or a layered structure having pores, openings, and/or interconnected void spaces that permit liquid water, urine, or synthetic urine to pass through its thickness in the absence of a forcing pressure. Conversely, the term “water-impermeable” refers to a layer or a layered structure through the thickness of which liquid water, urine, or synthetic urine cannot pass in the absence of a forcing pressure (aside from natural forces such as gravity). A layer or a layered structure that is water-impermeable according to this definition may be permeable to water vapor, i.e., may be “vapor-permeable”.

“Extendibility” and “extensible” mean that the width or length of the component in a relaxed state can be extended or increased.

“Elasticated” and “elasticized” mean that a component comprises at least a portion made of elastic material.

“Elongatable material”, “extensible material”, or “stretchable material” are used interchangeably and refer to a material that, upon application of a biasing force, can stretch to an elongated length of at least about 110% of its relaxed, original length (i.e. can stretch to 10 percent more than its original length), without rupture or breakage, and upon release of the applied force, shows little recovery, less than about 20% of its elongation without complete rupture or breakage as measured by EDANA method 20.2-89. In the event such an elongatable material recovers at least 40% of its elongation upon release of the applied force, the elongatable material will be considered to be “elastic” or “elastomeric.” For example, an elastic material that has an initial length of 100 mm can extend at least to 150 mm, and upon removal of the force retracts to a length of at least 130 mm (i.e., exhibiting a 40% recovery). In the event the material recovers less than 40% of its elongation upon release of the applied force, the elongatable material will be considered to be “substantially non-elastic” or “substantially non-elastomeric”. For example, an elongatable material that has an initial length of 100 mm can extend at least to 150 mm, and upon removal of the force retracts to a length of at least 145 mm (i.e., exhibiting a 10% recovery).

“Dimension”, “Length”, “Width”, “Pitch”, “Diameter”, “Aspect Ratio”, “Angle”, and “Area” of the article are all measured in a state wherein the article is extended to the Full Stretch Circumference W1 according to the “Whole Article Force Measurement” herein, and utilizing a ruler or a loupe, unless specified otherwise.

“Basis weight” of a nonwoven substrate or other material is measured by EDANA method 20.2-89.

“Artwork” refers to a visual presentation to the naked eye, which is provided by printing or otherwise, and having a color. Printing includes various methods and apparatus well known to those skilled in the art such as lithographic, screen printing, flexographic, and gravure ink jet printing techniques.

“Color” or “Colored” as referred to herein includes any primary color except color white, i.e., black, red, blue, violet, orange, yellow, green, and indigo as well as any declination thereof or mixture thereof. The color white is defined as those colors having a L* value of at least 94, an a* value equal to 0 ± 2, and a b* value equal to 0 ± 2 according to the CIE L* a* b* color system.

### DETAILED DESCRIPTION

### Absorbent Article

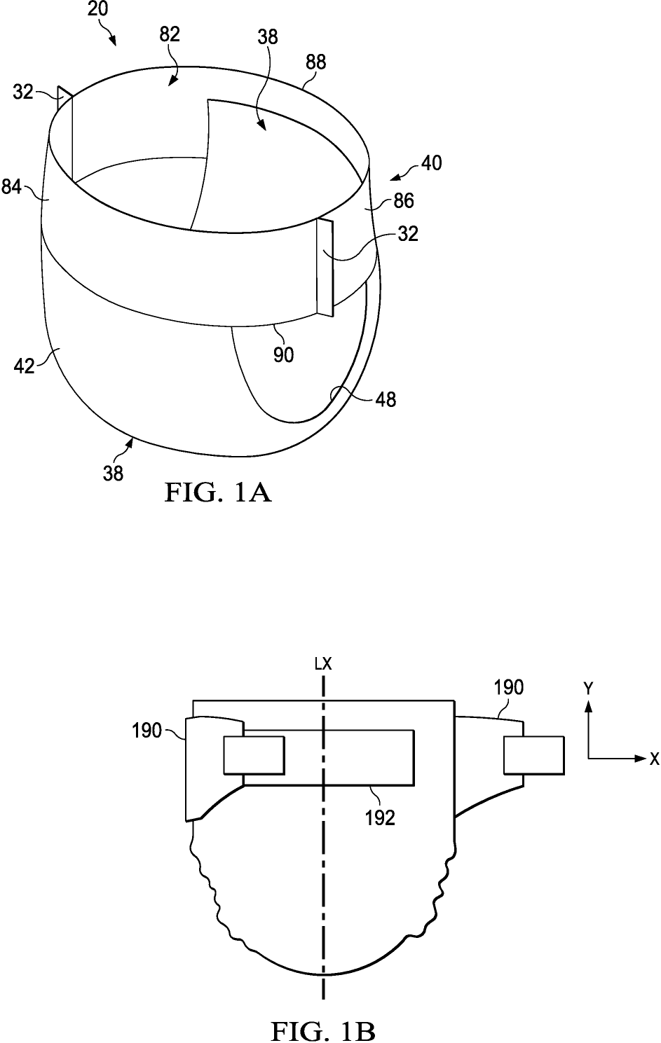

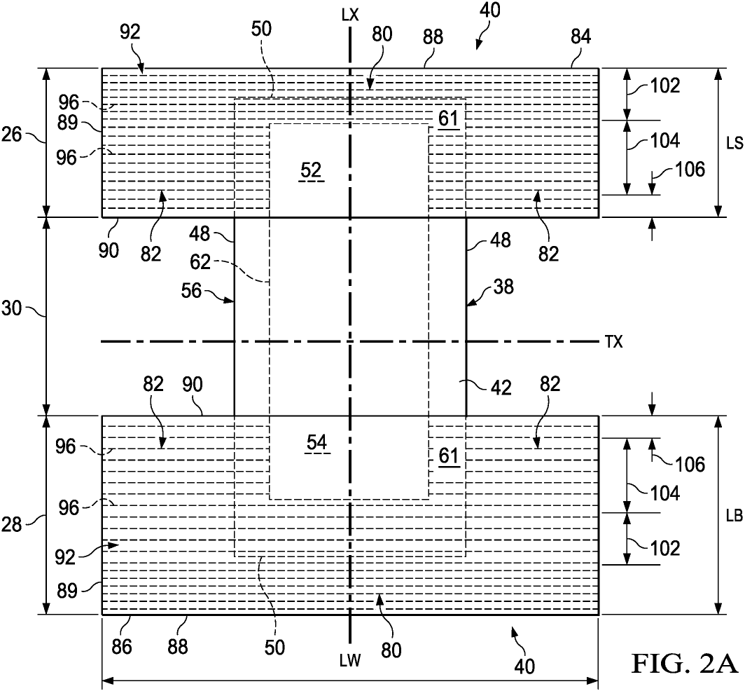

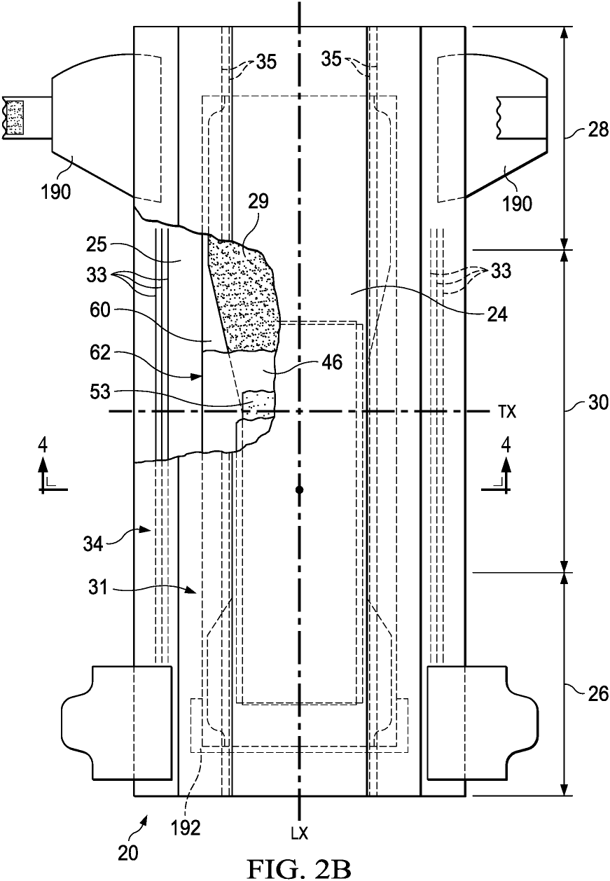

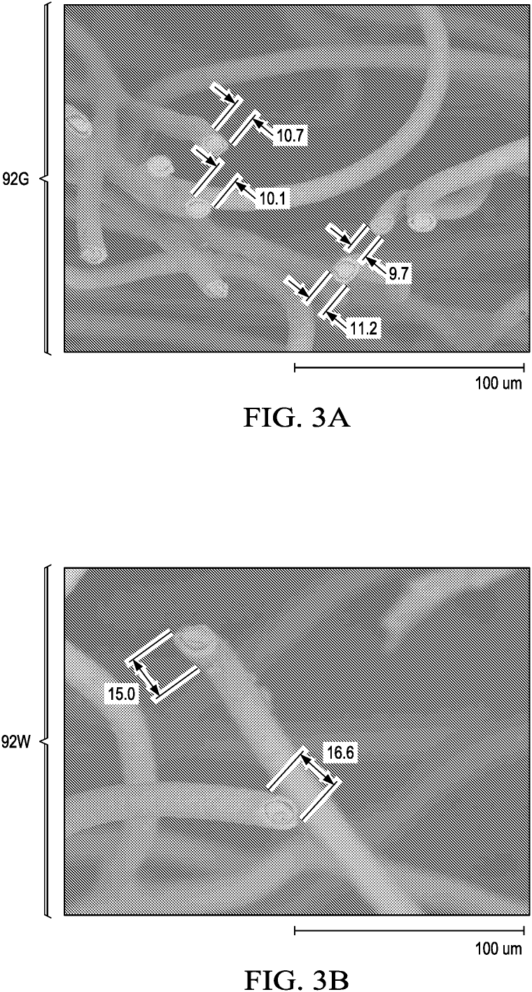

FIG. 1A is a perspective view of a pant type absorbent article (20) of the present invention, and FIG. 2A is a schematic plan view of such an absorbent article with the seams unjoined and in its flat uncontracted condition showing the garment-facing surface. FIG. 1B is a schematic perspective view of a taped type absorbent article (20) of the present invention, and FIG. 2B is a schematic plan view of such an absorbent article in its flat uncontracted condition showing the wearer-facing surface. The absorbent article (20) has a longitudinal centerline LX which also serves as the longitudinal axis, and a transverse centerline TX which also serves as the transverse axis. The absorbent article (20) has a body facing surface, a garment facing surface, a liquid permeable topsheet, a liquid impermeable backsheet, an absorbent core disposed between the topsheet and the backsheet, and an outer cover layer for covering the garment-facing side of the backsheet. The absorbent article of the present invention may be assembled together with an application means, wherein the application means is selected from the group of a fastening means and an elastic belt.

The application means may be an elastic belt, and the absorbent article (20) may take the form of a belt-type pant as in FIGS. 1A and 2A comprising a central chassis (38) to cover the crotch region (30) of the wearer, a front elastic belt (84) and a back elastic belt (86) (hereinafter may be referred to as “front and back elastic belts”), the front and back elastic belts (84, 86) forming a discrete ring-like elastic belt (40) extending transversely defining the waist opening. For the belt-type pant, the discrete ring-like elastic belt (40) may also be referred to as the elastic belt (40). For the belt-type pant as in FIGS. 1A and 2A, the front and back elastic belts (84, 86) and the central chassis (38) jointly define the leg openings. For the belt-type pant, the front elastic belt (84) is the front region (26), and the back elastic belt (86) is the back region (28), and the remainder is the crotch region (30). While not shown, the absorbent article (20) may be a uni-body type pant configured such that the outer cover of the central chassis (38) and the elastic belt (40) are common. For the uni-body type pant, the portion extending in the transverse direction between the side seams (32), respectively, are considered the front region (26) and the back region (28), and the remainder is the crotch region (30). For the uni-body type pant, the front region (26) is considered the front elastic belt region (84), and the back region (28) is considered the back elastic belt region (86).

The central chassis (38) may comprise a liquid permeable topsheet (24), a liquid impermeable backsheet (25) and an absorbent core (62) disposed between the topsheet (24) and the backsheet (25), and further an outer cover layer (42) for covering the garment-facing side of the backsheet (25). The outer cover layer (42) may be a nonwoven sheet. The central chassis (38) may contain an absorbent core (62) for absorbing and containing body exudates disposed on the central chassis (38), and an absorbent material non-existing region (61) surrounding the periphery of the absorbent core (62). The absorbent material non-existing region (61) may be made of the topsheet (24) and/or the backsheet (25) and/or the outer cover layer (42) and/or other parts configuring the central chassis (38). In the embodiment shown in FIG. 2A, the central chassis (38) has a generally rectangular shape, left and right longitudinally extending side edges (48) and front and back transversely extending end edges (50). The absorbent core (62) may exist through the entire longitudinal dimension of the crotch region and extending at least partly in the front region (26); or at least partly in both the front and back regions (26, 28). The central chassis (38) may have a front waist panel (52) positioned in the front region (26) of the absorbent article (20), a back waist panel (54) positioned in the back region (28), and a crotch panel (56) between the front and back waist panels (52, 54) in the crotch region (30). The center of the front elastic belt (84) is joined to a front waist panel (52) of the central chassis (38), the center of the back elastic belt (86) is joined to a back waist panel (54) of the central chassis (38), the front and back elastic belts (84, 86) each having a left side panel and a right side panel (82) where the central chassis (38) does not overlap. The central chassis has a crotch panel (56) positioned between the front waist panel (52) and the back waist panel (54).

The application means may be a fastening system, and the absorbent article (20) may take the form of a taped type. Referring to FIGS. 1B and 2B, the absorbent article may comprise a fastening system comprising a pair of elongate members (190) and a receiving member (192), the elongate members (190) transversely protruding from the left and right side edges of the back region and fastenable with the receiving member (192) disposed on the front region. Alternatively, the elongate members (190) may be protruding from the front region and fastenable with the receiving member (192) on the back region. The elongate members (190) may comprise an attaching portion, an extending portion, and refastenable means. The extending portion may be made of highly stretchable laminate for receiving stretching force upon applying the absorbent article, and the refastenable means may be made of material physically engageable with materials of the receiving member (192). The combination of materials useful for the refastenable means and the receiving member (192) include hook and loop, latch and hole, button and hole, hook and hole, low tackifying adhesive agent, and combinations thereof. The receiving member (192) may also have a protruding portion which may or may not be equipped with refastenable means.

### Outer Cover Layer

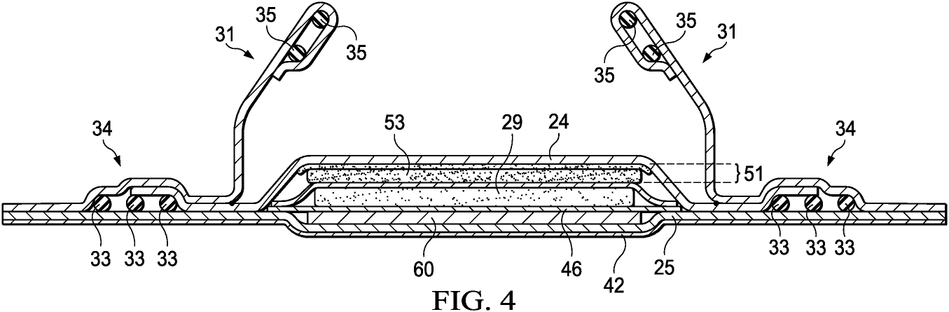

Referring to FIG. 4, the absorbent article of the present invention comprises an outer cover layer (42) forming a relatively major area of the garment-facing side of the article. The outer cover layer (42) thus may be closely associated with the function and quality of the article. Thus, materials for forming the outer cover layer (42) is carefully selected by the manufacturer to provide the desired tactile and visible senses. Tactile sense such as flexibility and cushiony touch may enhance perception of high quality. For providing such favorable tactile sense, the outer cover layer (42) of the present invention comprises a garment facing surface and a wearer facing surface, the outer cover layer (42) formed by a muti-fiber layer nonwoven (MLN) having a basis weight of from about 16gsm to about 35gsm and comprising a garment facing layer (92G) and a wearer facing layer (92W), the garment facing layer (92G) comprising fibers having a diameter of about 11 µm or less, preferably from about 7 µm to about 11 µm, and the wearer facing layer (92W) comprising fibers having a diameter of about 13 µm or more, preferably from about 13 µm to about 24 µm, wherein the weight ratio of the garment facing layer (92G) is from about 20% to about 70% of the multi-fiber layer nonwoven (MLN).

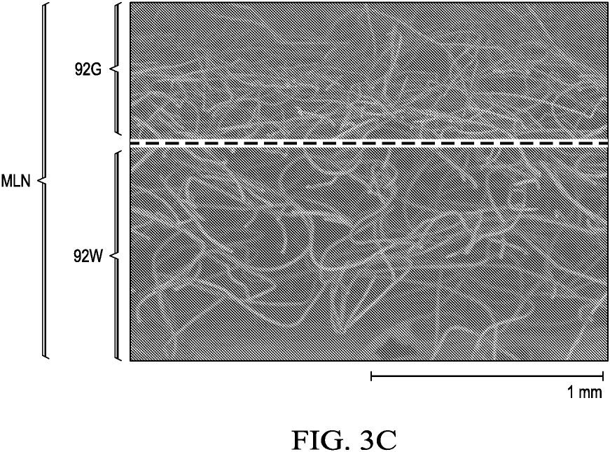

By multi-fiber layer nonwoven (MLN) herein, what is meant is a nonwoven comprising distinct layers in the thickness direction of fibers of different diameter size. Referring to FIG. 3A, the multi-fiber layer nonwoven (MLN) may be made of 2 layers, namely the garment facing layer (92G) and the wearer facing layer (92W), or may be made of more than 2 layers, having an additional layer between the garment facing layer (92G) and the wearer facing layer (92W). The garment facing layer (92G) comprises fibers having a diameter of about 11 µm or less, preferably from about 7 µm to about 11 µm, as measured according to the method herein. Fibers of such fineness are believed to provide a very smooth tactile sense when touched, as the fibers are below distinctive perceivable distance of tactile sensory to the human skin. The wearer facing layer (92W) comprising fibers having a diameter of about 13 µm or more, preferably from about 13 µm to about 24 µm, as measured according to the method herein. The fibers of the wearer facing layer (92W) provide some structural strength and cushiony tactile sense. Without being bound by theory, by providing the multi-fiber layer nonwoven (MLN) to have a basis weight of from about 16gsm to about 35gsm and a weight ratio of the garment facing layer (92G) of from about 20% to about 70%, a nonwoven material having balanced softness attributes such as smoothness, cushiony feel, and lack of grains/neps/lumps feeling is provided. The measurement for obtaining the diameter of the fibers are provided in further detail below.

The muti-fiber layer nonwoven (MLN) for forming the outer cover layer (42) may be made by processes such as spunbond, spunlace, carded or air-laid; and may comprise fibers and/or filaments made of polypropylene (PP), polyethylene (PE), polyethylene phthalate (PET), polylactic acid/polylactide (PLA) or conjugate fibers (such as PE/PET, PE/PP, PE/PLA) as well as natural fibers such as cotton or regenerated cellulosic fibers such as viscose or lyocell. The outer cover layer (42) nonwoven may be a multilayer or composite structure combining nonwovens made by different processes and fibers such as combining spunbond and carded nonwovens. The outer cover layer (42) nonwoven may be made by biodegradable material, or derived from renewable resources. Exemplary material for the outer cover layer (42) include: air-through carded nonwoven having a thickness of at least about 50 µm, or at least about 80 µm, or at least about 200 µm. Such material may provide a soft lofty feeling to the garment-facing side. Suitable for the outer cover layer (42) nonwoven of the present invention are air-through carded nonwoven material made of co-centric bicomponent fiber, crimping fiber made through core eccentric bicomponent filament or side by side bicomponent filament. One non-limiting material for the multi-fiber layer nonwoven (MLN) is a bicomponent fiber made of PE sheath and PET core which is airlaid. When such material is utilized, the fibers of the garment facing layer (92G) may be from about 0.6 to about 0.8 denier, and the fibers of the wearer facing layer (92W) may be from about 1.0 to about 2.0 denier. Non-limiting examples of commercially available materials suitable for the outer cover layer (42) nonwoven of the present invention include: 16-35gsm air-through carded nonwoven substrate comprising PE/PET bi-component fibers, such as those available from Jiangsu Wisdom Nonwoven Co. Ltd. or Xiamen Yanjan New Material Co. Ltd.

### Absorbent Core

Referring to FIG. 4, the absorbent article (20) of the present invention comprises an absorbent core (62) for absorbing and containing body exudates disposed on the wearer facing side. The absorbent core (62) may include an absorbent layer and an acquisition system (51). The absorbent layer is the region wherein absorbent materials (29) having a high retention capacity, such as superabsorbent polymers, are present. The absorbent layer may be substantially cellulose free. Alternatively, the absorbent layer may contain cellulose. There may be an absorbent layer mainly comprising cellulose, and another absorbent layer mainly comprising superabsorbent polymers.

Superabsorbent polymers of the absorbent layer may be disposed between first and second layers of material immobilized by a fibrous layer of thermoplastic adhesive material. The first and second layers of materials may be nonwoven fibrous webs including synthetic fibers, such as mono-constituent fibers of PE, PET and PP, multiconstituent fibers such as side by side, core/sheath or island in the sea type fibers. Such synthetic fibers may be formed via a spunbonding process or a meltblowing process. The acquisition system (51) facilitates the acquisition and the distribution of body exudates and may be placed between the topsheet (24) and the absorbent layer. The acquisition system (51) may include cellulosic fibers.

The absorbent layers may be disposed in plurality in the absorbent core (62). Some portions of the absorbent layers may be configured to have substantially no absorbent material to form a channel or a plurality of channels. Channels may be useful for allowing the absorbent core (62) to bend upon swelling with fluids, such that the crotch region conforms to the wearer’s body after swelling and prevent sagging of the article. The channels may also be formed in the acquisition system (51), and may be configured to at least partly match the channels of the absorbent layer in the thickness direction.

The absorbent core (62) may comprise a high loft material encompassing superabsorbent polymers. The term “high loft” refers to low density bulky fabrics, as compared to flat, paper-like fabrics. High loft webs are characterized by a relatively high porosity. This means that there is a relatively high amount of void space in which superabsorbent polymer particles can be distributed. The high loft material (without the superabsorbent particles) of the invention may have a density at a pressure of 4.14 kPa (0.6 psi) below 0.20 g/cm³, in particular ranging from 0.05 g/cm³ to 0.15 g/cm³. The high loft layer (without the superabsorbent particles) may have a density at a pressure of 2.07 kPa (0.3 psi) below 0.20 g/cm³, in particular ranging from 0.02 g/cm³ to 0.15 g/cm³. The high loft layer (without the superabsorbent particles) of the invention may have a density at a pressure of 0.83 kPa (0.12 psi) below 0.15 g/cm³, in particular ranging from 0.01 g/cm³ to 0.15 g/cm³, and a basis weight of from 15 to 500gsm, preferably 30~200gsm, such as those described in US 2021/0361497 A1. The absorbent core (62) comprising high loft material encompassing superabsorbent polymers may also contain channels.

Alternatively, the absorbent core (62) may comprise an absorbent layer having superabsorbent polymers disposed between first and second layers of nonwoven material immobilized by a fibrous layer of thermoplastic adhesive material (not shown). The first and second layers of nonwoven materials may be relatively low basis weight nonwoven fibrous webs including synthetic fibers, such as mono-constituent fibers of PE, PET and PP, multiconstituent fibers such as side by side, core/sheath or island in the sea type fibers. Such synthetic fibers may be formed via a spunbonding process or a meltblowing process. Such an embodiment is exemplarily shown in FIG. 4. In such embodiments, a) the intermediate layer (60) may be hydrophobic and the lower substrate layer (46) may be hydrophilic; or b) the intermediate layer (60) and the lower substrate layer (46) may both be hydrophilic and the intermediate layer (60) may be less hydrophilic than the lower substrate layer (46); or c) the intermediate layer (60) and the lower substrate layer (46) may both be hydrophobic and the lower substrate layer (46) may be less hydrophobic than the intermediate layer (60).

The absorbent core (62) may further comprise a liquid management layer (53) directly under the topsheet (24). The liquid management layer (53) may be part of the acquisition system (51). The function of such a layer is to rapidly acquire the fluid from the topsheet (24) away from the wearer-facing side and/or to distribute over a larger area so it is more efficiently absorbed by the absorbent core. It is also possible that such a liquid management layer (53) may be placed between the backsheet (25) and the absorbent core. The liquid management layer may be a spunlace nonwoven comprising viscose, PET, CoPET/PET fibers, and combinations thereof.

### Intermediate Layer

The absorbent core (62) may comprise an intermediate layer (60) between the layer of absorbent material and the backsheet (25). The intermediate layer (60) may be in direct contact with the layer of absorbent material (29) and with the backsheet (25). The intermediate layer (60) may be useful as a masking layer to isolate the superabsorbent polymer particles in the layer of absorbent material from the backsheet (25), thus reducing graininess feeling and improving the tactile properties of the garment-facing side of the article, especially for absorbent core (62) containing a high level of superabsorbent polymer particles.

The intermediate layer (60) may further isolate the exudates which have been absorbed in the layer of absorbent material from the garment-facing side of the article, as this may be visually unpleasant to the caregiver. Thus by having an intermediate layer with a relatively high opacity, stains in the layer of absorbent material (e.g. from urine or feces) can be concealed from view, when looking at the backsheet (25) of the absorbent article during use. The dry opacity of the intermediate layer may be at least 25%, or at least 40%, or at least 50%, or at least 70%. The intermediate layer (60) can also help reduce the residual moisture in contact with the backsheet (25), which may lead to cold/wet feeling for the caregiver, or may lead to the wearer mistaking the cold/wet feeling as liquid leaking out of the absorbent article. The intermediate layer (60) may also serve as a temporary reservoir for liquid that had not been absorbed fast enough by the layer of absorbent material.

Additional layers provided to an absorbent core (62) generally increase the thickness and bulkiness of the article. This may lead to increased bending stiffness in the crotch region, thus acting as drawback for conformity and close contact of the article to the wearer’s body, thereby reducing wearer comfort. Therefore, it is desirable for the intermediate material (60) to have a thickness that can survive compressive force, while also having a cushiony benefit even when compressed. Accordingly, the intermediate layer may have an MD Tensile/Basis Weight of no greater than about 0.75 N/5 cm/g/m² or greater than about 0.71 N/5 cm/ g/m² as measured according to measurements herein, and a Thickness/Basis Weight of no less than about 0.078 mm/g/m², or no less than about 0.80 mm/g/m², or no less than about 0.90 mm/g/m² as according to measurements herein. Lower values for MD Tensile/Basis Weight indicate that a material is less bonded and more flexible versus higher values. Higher values for Thickness/Basis Weight indicate that a material is loftier under compression versus lower values.

The basis weight of the intermediate layer (60) may be homogeneous throughout longitudinal and transverse direction of the intermediate layer (60). The intermediate layer (60) may have a smaller extension in the longitudinal and/or transverse direction than the layer of absorbent material, such that absorbent material (29) extends beyond the intermediate layer in the longitudinal and/or transverse direction. Alternatively, the intermediate layer (60) may have a larger extension in the longitudinal and/or transverse direction than the absorbent material (29) when the absorbent layer is in direct contact with the intermediate layer.

The intermediate layer (60) may be a carded air-through bonded nonwoven, a carded calendar bonded nonwovens nonwoven web, a spunbond or meltblown nonwoven web (made of continuous fibers) or a nonwoven with spunbond and meltblown layers (e.g. an SMS, SMMS, SMSS or the like). In one embodiment, the intermediate layer (60) is a carded air-through bonded nonwoven. The nonwoven web may be made of synthetic fibers, such as polyolefin (e.g. polyethylene, polypropylene or mixtures or combinations thereof), polyethylene terephthalate (PET), co-PET, polylactic acid (PLA), polyhydroxy alkanoid (PHA), or combinations or mixtures thereof. The fibers may be continuous or staple fibers.

The intermediate layer (60) may comprise a nonwoven web comprising first thermoplastic fibers having a first melting temperature and second thermoplastic fibers having a second melting temperature, a difference of the first melting temperature and the second melting temperature is at least about 40° C., or at least 50° C., or at least 60° C. If melting temperature of different fiber types get close more or all fiber types will bond to each other and/or to itself which will result in excessive stiffness which is not desired. When the first thermoplastic fiber comprises at least two polymers having different melting temperatures, a melting temperature of a polymer lower than melting temperature(s) of any other polymer(s) constituting the first thermoplastic fiber is considered the first melting temperature. By the same token, when the second thermoplastic fiber comprises at least two polymers having different melting temperatures, one melting temperature of a polymer lower than melting temperature(s) of any other polymer(s) constituting the second thermoplastic fiber is considered the second melting temperature.

In the embodiment where the nonwoven web comprised by or forming the intermediate layer comprises first thermoplastic fibers having a first melting temperature and second thermoplastic fibers having a second melting temperature, a difference of the first melting temperature and the second melting temperature is at least about 40° C., the nonwoven web may comprise at least 40 wt%, or at least 50 wt%, or at least 60 wt% of the first or the second thermoplastic fibers whichever having a lower melting temperature based on the total weight of the nonwoven web.

In the embodiment where the nonwoven web comprised by or forming the intermediate layer comprises first thermoplastic fibers having a first melting temperature and second thermoplastic fibers having a second melting temperature, a difference of the first melting temperature and the second melting temperature is at least about 40° C., the nonwoven web may comprise at least 30 wt%, or at least 40 wt%, or at least 50 wt% of the first or the second thermoplastic fibers whichever having a higher melting temperature based on the total weight of the nonwoven web.

In the embodiment where the nonwoven web comprised by or forming the intermediate layer comprises first thermoplastic fibers having a first melting temperature and second thermoplastic fibers having a second melting temperature, a difference of the first melting temperature and the second melting temperature is at least about 40° C., fibers having a lower melting temperature may be heat-fused one another, and/or substantial part of fibers having a higher melting temperature may not heat-fused one another.

In one embodiment when the second thermoplastic fibers have a melting temperature greater at least about 40° C. than the first thermoplastic fibers, the first thermoplastic fibers having a melting temperature lower than the second thermoplastic fibers, hollow fibers in this case, in the nonwoven web are heat-fused one another. The presence of the first thermoplastic fibers which are not heat-fused one another is acceptable as long as majority of the first thermoplastic fibers are heat-fused one another. The second thermoplastic fibers, hollow fibers in this case, in the nonwoven web are not heat-fused one another. Further, majorities of the first thermoplastic fibers and the second thermoplastic fibers may not be heat-fused each another.

Without being bound by theory, optimizing fiber to fiber bonding per mass of nonwoven web may enable the intermediate layer to have a high thickness under compression and a low stiffness especially in the crotch. Increase of fiber to fiber bonding in the nonwoven may increase the stiffness of the material. On the other hand, decrease of fiber-to-fiber bonding in nonwoven web may result in less integrity of the nonwoven which is more prone to collapse of the material under compressive forces.

The first thermoplastic fiber may be a solid round fiber, a hollow fiber or a shaped fiber. The second thermoplastic fiber may be a solid round fiber, a hollow fiber or a shaped fiber. In one embodiment, the second thermoplastic fiber is a hollow fiber or a shaped fiber. In the embodiment, the second thermoplastic fiber may be hollow conjugate fiber.

Shaped fibers also may introduce higher specific surface area which increases the capillary pressure of the second web layer containing shaped fibers which can lead to better drainage of the first web layer by the second fiber web layer comprising shape fibers. Shaped fibers may include bilobal shaped, trilobal shaped, quatro-lobal shaped, delta shaped, concave delta shaped, crescent shaped, oval shaped, star shaped, square shaped, U-shaped, H-shaped, C-shaped, V-shaped, diamond shaped fibers.

Hollow fibers enable greater loft with larger effective diameter per linear density with less weight. They also provide better resilience under compression. Hallow fibers can be hollow conjugate fibers with spiral and/or 3D crimp to maximize the benefits of loft and resilience. Such hollow conjugate fibers can have non-uniform properties across the fiber cross-section for instance by using polymers with different characteristics (e.g. different polymers or same polymer with different characteristics such as viscosity).

Without being bound by theory, hollow fibers or shaped fibers may be advantageous over solid round fibers to provide improved cushiony characteristics as hollow fibers and shaped fibers have higher resilience at the same fiber denier due to having higher effective radius compared to round fibers.

Each of the first and the second thermoplastic fibers may be monocomponent fibers or multicomponent fibers, such as bicomponent fibers. If the fibers are bicomponent fibers, they have a core-sheath configuration, wherein the core component has a higher melting temperature than the sheath component.

The intermediate layer comprises or consists of a nonwoven web which is air-through bonded. Such nonwoven webs generally have high loft. Hence, they have a porous structure to provide void volume for absorbing and temporarily holding liquid. At the same time, they provide softness and do not have an excessively high bending stiffness.

The fibers may be continuous, such as in a spunlaid nonwoven web. The spunlaid nonwoven web is preferably air-through bonded or spunlace. In addition to hydroentanglement (spunlace) or air-through bonding, the spunlaid nonwoven web may or may not have undergone some localized bonding with heat and/or pressure (e.g. point bonding), introducing localized bond regions where the fibers are fused to each other.

In some embodiments, the fibers comprised by the intermediate layer are staple fibers. Similar to a nonwoven web made of continuous fibers, a nonwoven web of staple fibers is preferably carded nonwoven such as air-through bonding nonwoven. In addition to air-through bonding, the nonwoven web of staple fibers may or may not have undergone some localized bonding with heat and/or pressure (e.g. point bonding), introducing localized bond regions where the fibers are fused to each other.

Irrespective whether the nonwoven web is made of continuous fibers or staple fibers, the localized bonding should however not bond an excessively large surface area, thus negatively impacting the loft and void volume of the nonwoven web as well as stiffness. Preferably, the total bond area obtained by localized bonding with heat and/or pressure (in addition to hydroentanglement or air-through bonding) should not be more than 20%, or not be more than 15%, or not be more than 10% of the total surface area of the nonwoven web.

Alternatively, the nonwoven web comprised by the intermediate layer should not have undergone any bonding and consolidation in addition to the hydroentanglement (spunlace) or air-through bonding. Thereby, the advantageous properties of such nonwoven webs can be used to their optimum.

Alternatively, the nonwoven web comprised by the intermediate layer should not have undergone any bonding and consolidation in addition to the hydroentanglement (spunlace) or air-through bonding. Thereby, the advantageous properties of such nonwoven webs can be used to their optimum.

In a spunlace nonwoven web the fibers have been subjected to hydroentanglement to intermingle and intertwine the fibers with each other. Cohesion and the interlacing of the fibers with one another may be obtained by means of a plurality of jets of water under pressure passing through a moving fleece or cloth and, like needles, causing the fibers to intermingle with one another. Thus, consolidation of a spunlace nonwoven web is essentially a result of hydraulic interlacing. “Spunlace nonwoven web”, as used herein, also relates to a nonwoven formed of two or more precursor webs, which are combined with each other by hydraulic interlacing. The two or more webs, prior to being combined into one nonwoven by hydraulic interlacing, may have underdone bonding processes, such as heat and/or pressure bonding by using e.g. a patterned calendar roll and an anvil roll to impart a bonding pattern. However, the two or more webs are combined with each other solely by hydraulic interlacing. Alternatively, the spunlace nonwoven web is a single web, i.e. it is not formed of two or more precursor webs. Spunlace nonwoven layers/webs can be made of staple fibers or continuous fibers.

Through-air bonding (interchangeably used with the term “air-through bonding”) means a process of bonding staple fibers or continuous fibers by forcing air through the nonwoven web, wherein the air is sufficiently hot to melt (or at least partly melt, or melt to a state where the fiber surface becomes sufficiently tacky) the polymer of a fiber or, if the fibers are multicomponent fibers, wherein the air is sufficiently hot to melt (or at least partly melt, or melt to a state where the fiber surface becomes sufficiently tacky) one of the polymers of which the fibers of the nonwoven web are made. The air velocity is typically between 30 and 90 meter per minute and the dwell time may be as long as 6 seconds. The melting and re-solidification of the polymer provide the bonding between different fibers.

### Other Components

Referring to FIG. 4, the absorbent article (20) of the present invention may further comprise components that improve the fit of the article around the legs of the wearer, in particular barrier leg cuffs (31) and gasketing leg cuffs (34). The barrier leg cuffs (31) may be formed by a piece of material, typically a nonwoven, which is partially bonded to the rest of the article and can be partially raised away and thus stand up from the plane defined by the topsheet (24). The barrier leg cuffs (31) are typically delimited by a proximal edge joined to the rest of the article, typically the topsheet (24) and/or the backsheet (25), and a free terminal edge intended to contact and form a seal with the wearer’s skin. The standing up portion of the cuffs typically comprise an elastic element, for example one or a plurality of elastic strands (35). The barrier leg cuffs (31) provide improved containment of liquids and other body exudates approximately at the junction of the torso and legs of the wearer.

In addition to the barrier leg cuffs (31), the article may comprise gasketing leg cuffs (34), which may be at least partially enclosed between the topsheet (24) or the barrier leg cuffs (31) and the backsheet (25), and may be placed laterally outwardly relative to the upstanding barrier leg cuffs (31). The gasketing leg cuffs (34) can provide a better seal around the thighs of the wearer. Usually each gasketing leg cuff (34) will comprise one or more elastic string or elastic element (33) comprised in the chassis of the diaper for example between the topsheet (24) and backsheet (25) in the area of the leg openings.

### Elastic Belt

The pant type absorbent article of the present invention may comprise an elastic belt (40) which acts to dynamically create fitment forces and to distribute the forces dynamically generated during wear. The front and back elastic belts (84, 86) may be joined with each other only at the side edges (89) to form side seams (32), a waist opening and two leg openings. Each leg opening may be provided with elasticity around the perimeter of the leg opening. The elasticity around the leg opening may be provided by the combination of elasticity from the front belt (84), the back belt (86), and the leg cuffs (31, 34).

The front elastic belt (84) and back elastic belt (86) are configured to impart elasticity to the belt (40). Referring to FIGS. 1A and 2A, the front belt (84) and the back belt (86) may each comprise a laminate, the laminate comprising an outer sheet (92), an inner sheet (94), and a plurality of elastic members (96) running in the transverse direction. The elastic belt region (40) may be closely associated with the function and quality of the article. Thus, materials for forming the elastic belt region (40), as well as the gathering profile of the elastic belt region, are carefully selected by the manufacturer to provide the desired tactile and visible senses. The outer sheet (92) may be formed by the muti-fiber layer nonwoven suitable for forming the outer cover layer (42). The outer cover layer (42) and outer sheet (92) may be formed by the same muti-fiber layer nonwoven material. By providing the entire garment-facing surface of the absorbent article by the same muti-fiber layer nonwoven material, an absorbent article having overall improved softness connoting high quality may be obtained.

The inner sheet (94) for making the laminate may be a nonwoven having a basis weight of from about 5gsm to about 45gsm, or from about 5gsm to about 35gsm. The inner sheet (94) nonwoven may have a fiber diameter of from about 0.5 dpf to about 4 dpf. The inner sheet (94) nonwoven may be made by processes such as spunbond, spunlace, carded or air-laid; and may comprise fibers and/or filaments made of polypropylene (PP), polyethylene (PE), polyethylene phthalate (PET), polylactic acid/polylactide (PLA) or conjugate fibers (such as PE/PET, PE/PP, PE/PLA) as well as natural fibers such as cotton or regenerated cellulosic fibers such as viscose or lyocell. The inner sheet (94) nonwoven may also be a multilayer or composite structure combining nonwovens made by different processes and fibers such as combining spunbond and carded nonwovens. The inner sheet (94) nonwoven may be made by biodegradable material, or derived from renewable resources. Non-limiting examples of materials suitable for the inner sheet (94) nonwoven of the present invention include: 8-45gsm spun melt nonwoven substrate comprising PP monofilament or PE/PP bi-component fibers, such as those available from Malaysia Fibertex, Avogl China, 12-30gsm air-through carded nonwoven substrate made of PE/PET bi-component staple fiber, such as those available from Beijing Dayuan Nonwoven Fabric Co. Ltd. or Xiamen Yanjan New Material Co. Ltd., and 8-30gsm spun melt nonwoven substrate comprising PP monofilament or PE/PP bi-component fibers, such as those available from Fibertex or Fitesa.

The basis weight of the outer sheet (92) and the inner sheet (94) may be adjusted such that the basis weight of the inner sheet (94) is not greater than the basis weight of the outer sheet (92). Thus, the outer sheet (92) may be provided with a soft lofty tactile sense which connotes high quality, while the inner sheet (94) may be kept thinner and conforming to the outer sheet (92), thus saving cost. Further, without being bound by theory, by providing the basis weight relationship as such, it is believed that skin sweating is effectively transported to the outer sheet (92) and outside the laminate, while preventing the transported sweat back to the inner sheet (94). The hydrophilicity/hydrophobicity of the outer sheet (92) and the inner sheet (94) is adjusted such that the hydrophilicity of the outer sheet (92) is higher than that of the inner sheet (94). Without being bound by theory, it is believed that such gradient of hydrophilicity is advantageous in transporting skin sweat from the inner sheet (94) to the outer sheet (92) and outside the laminate. The inner sheet (94) nonwoven may be inherently hydrophobic. The inner sheet (94) nonwoven may be provided hydrophobicity by treating with hydrophobic melt additives into polymer resin in the fiber making process, or by applying hydrophobic additives after the nonwoven is formed. The outer sheet (92) nonwoven may inherently be hydrophobic, and thus provided relatively more hydrophilic than the inner sheet (94) by treating with hydrophilic melt additives into polymer resin in the fiber making process, or by applying hydrophilic additive after the nonwoven is formed.

Referring to FIG. 2A, the elastic member (96) may be made by a plurality of elastic strands (96) running parallel to each other in the transverse direction, wherein the laminate has a region wherein the elastic strands (96) have a longitudinal pitch of from about 3 mm to about 18 mm, or from about 3 mm to about 12 mm, or from about 3 mm to about 7 mm.

The tensile stress (N/m) of the entirety of the front and back elastic belts (84, 86), respectively, may be profiled in order to provide the functional benefits of the present invention, such as ease of stretch and application, while also maintaining certain force during wear, to prevent the article from sagging after loading. When the elasticity of the front and back elastic belts (84, 86) are provided by a plurality of elastic members (96) running in the transverse direction, the tensile stress may be adjusted by one or more of the following methods; 1) elongation rate of the elastic member (96); 2) density (dtex) of the elastic member (96); 3) longitudinal pitch of multiple elastic members (96); and 4) effective length of elasticity of the elastic member (96) in the transverse direction. By elongation, “0% elongation” is meant the original length of the elastic member. When a portion of an elastic member (96) is removed of its elasticity, the remainder of the intact elastic member capable of imparting elasticity is defined as the “effective length of elasticity of an elastic member”.

Use of the multi-fiber layer nonwoven (MLN) for forming the outer sheet (92) is further advantageous as having a relatively high tolerance against tearing. This is believed to be due to relatively more bonding points provided to the nonwoven by utilizing fine fibers. Namely, due to the force applied to the side seam to tear open the side seam (32) in the lateral direction of the article, the substrate may rip in this direction. When the outer sheet (92) is made of the multi-fiber layer nonwoven (MLN), this is believed to prevent the material to ripping in an undesired direction when tear opening the side seam by hand along the longitudinal dimension for removal from the wearer.

The inner sheet (94) for forming the laminate may be a nonwoven made of material having a melting point of no more than about 165° C. By providing such inner sheet (94), the laminate formed together with the aforementioned outer sheet (92) may provide side seams (32) which tolerate normal usage conditions, while also being easy to open after use for removal.

### Laminate Bonding

As mentioned above, the elastic belt region (40) may be closely associated with the function and quality of the article. Thus, the gathering profile of the elastic belt region is also carefully selected by the manufacturer to provide the desired tactile and visible senses. Tactile sense such as flexibility and cushiony touch may enhance perception of high quality. The appearance of gathers may intuitively connote the function of the article, or the function of the elastic belt region (40). For example, relatively big uniform gathers may connote a fluffy and soft feel. For example, a bubble kind of texture may connote a soft and cushiony feel. Further, other functions provided by the laminate such as stretchability for ease of application, comfort and softness, as well as breathability, may enhance the perception provided by the gather appearance. Gathers intentionally provided to have a certain appearance may intuitively communicate the functional benefits described above, and provide the favorable entire usage experience of the article by the user. The user may be the wearer or the caregiver.

Referring to FIG. 2A, the front belt (84) and the back belt (86) may each comprise a laminate, the laminate comprising a plurality of elastic members (96) running in the transverse direction, an inner sheet (94), an outer sheet (92), and an outer sheet fold over (not shown) wherein the outer sheet fold over is an extension of the outer sheet material formed by folding the outer sheet material at the distal edge (88) of the front and back belts; wherein the belt elastic members (96) are sandwiched between two of these sheets. The longitudinal dimension between adjacent elastic members (96) form an elastic spacing. The front elastic belt (84) and the back elastic belt (86) may each be made only by elastic members (96), the inner sheet (94), the outer sheet (92), and the outer sheet fold over. The belt elastic members (96) may extend in the transverse direction to provide a ring like elastic belt (40) when the front elastic belt (84) and the back elastic belt (86) are joined. At least some of the elastic members (96) extend in the transverse direction substantially parallel to each other. All of the elastic members (96) may extend in the transverse direction substantially parallel to each other. Such an article may be economically made. The front and back elastic belt (84, 86) each may have transversely continuous proximal and distal edges, the proximal edge (90) being located closer than the distal edge (88) relative to the longitudinal center of the article. At least 10%, or at least from about 15% to not more than about 70%, of the front and back elastic belts from the waist opening in the longitudinal direction may be a laminate in active elasticity along the entire transverse dimension LW of the front and back elastic belts (84, 86). Referring to FIG. 2A, the front and back elastic belts (84, 86) may be treated such that certain regions are removed of its elastic activity to form a non-elastic region (221). For each front and back elastic belt (84, 86), the region overlapping the front and/or back waist panel (52, 54) of the central chassis (38) may be removed of its elastic activity and defining the non-elastic region (221).

The longitudinal length of the backsheet (25) and the outer cover layer (42) may be the same, or may be varied. For example, the outer cover layer (42) may have a shorter length compared to that of the backsheet (25), such that the outer cover layer (42) is devoid where the central chassis (38) overlaps the elastic belt (40). By such configuration, the elastic belt may have better breathability. Further, such configuration may provide cost saving. The transverse width of the backsheet (25) and the outer cover layer (42) may be the same, or may be varied. For example, the backsheet (25) may have a shorter transverse width compared to that of the outer cover layer (42). By such configuration, the longitudinal side edges (48) of the crotch panel (56), which make part of the leg openings, may have better breathability. Further, such configuration may provide cost saving.

### MEASUREMENT METHODS

### Basis Weight of Nonwoven Substrate

The basis weight of nonwoven substrates are measured according to “ISO 9073-1:1989 Textiles – Test methods for nonwovens - Part 1: Determination of mass per unit area”. To obtain the nonwoven sample, cut a rectangle-shaped nonwoven specimen from the article with an area of 100 cm² (for example, 100 mm × 100 mm), and measure its basis weight following the measurement principle used by the standard method above. The reported basis weight will be the average value of at least five replicates is reported to the nearest 1 gsm (g/m²).

### Fiber Diameter and Weight Ratio of Muti-Fiber Layer Nonwoven

### Nonwoven Sample Preparation and Conditioning

When available in its raw material form, a sample of a size of 5 ± 1 cm × 5 ± 1 cm is cut from the raw material. Otherwise, the nonwoven sample in the form of an outer sheet (92) or an outer cover layer (42) is removed from a finished wearable article. For the purpose of removing the nonwoven from the finished article, a razor blade is used to excise a sample of a size of 5 ± 1 cm × 5 ± 1 cm area from the underling layers of the article around the outer perimeter. (If the nonwoven is of insufficient size to permit a 5 ± 1 cm × 5 ± 1 cm area to be excised from the wearable article, the largest square of nonwoven that can be extracted is excised and used as the sample henceforth.) As necessary, cryogenic spray (such as Cyto-Freeze, Control Company, Houston TX) can be used to remove the nonwoven from the underling layer. The nonwoven samples obtained from a finished wearable article are pre-conditioned at ambient temperature and relative humidity (about 23° C. and 50% RH respectively) for at least 4 hours before measurements.

### Measurement of Fiber Diameter

To prepare the specimen for cross-section imaging, submerge the nonwoven sample obtained or removed from the wearable article in liquid nitrogen and use a razor blade to cut a 10 mm × 4 mm specimen from the nonwoven. The specimen is mounted vertically on a specimen stage with the wearer facing side attached onto the specimen stage using carbon tape. The cross-sectional edge of the specimen is facing upwards and oriented such that it is substantially aligned to the horizontal direction for subsequent imaging. The specimen is sputtered with platinum to avoid electric charging and improve overall conductivity, under the conditions of 30 mA current and 120 second coating time.

Cross section images of specimen are taken using a Scanning Electron Microscope (SEM) such as Tabletop Microscope TM3000 (Hitachi, Japan), or equivalent. The platinum-coated specimen is subsequently transferred into the SEM specimen vacuum chamber for the imaging analysis. An appropriate magnification and working distance are chosen so that the cross-section specimen is suitably enlarged for fiber diameter measurement and imaged under an acceleration voltage of 5kV. The specimen images are saved as 8-bit jpeg images containing a linear distance scale for calibration. Measurement of the fiber diameter is performed using an image analysis program such as ImageJ software (version 1.52p or above, National Institutes of Health, USA) or equivalent. Record the values of fiber diameter to the nearest 0.1 micron (as shown in FIGS. 3A-3C). Measure at least 10 replicates and report the average value to the precision described above.

### Measurement of Weight Ratio

### 1) Instrument Description

3D x-ray specimen imaging is obtained on a micro-CT instrument (a suitable instrument is the Scanco µCT 100 available from Scanco Medical AG, Switzerland, or equivalent). The micro-CT instrument is a cone beam microtomograph with a shielded cabinet. A maintenance free x-ray tube is used as the source with an adjustable diameter focal spot. The x-ray beam passes through the specimen, where some of the x-rays are attenuated by the specimen. The extent of attenuation correlates to the mass of material the x-rays have to pass through. The transmitted x-rays continue on to the digital detector array and generate a 2D projection image of the specimen. A 3D image of the specimen is generated by collecting several individual projection images of the specimen as it is rotated, which are then reconstructed into a single 3D image. The instrument is interfaced with a computer running software to control the image acquisition and save the raw data.

### 2) Image Acquisition

To prepare the specimen for 3D x-ray imaging, the nonwoven sample obtained or removed from the wearable article is die cut to obtain a circular piece with a diameter of 8 mm. Record the mass of the cut piece. Multiple specimens taken from the same specimen can be analyzed and compared to each other. Avoid folds, wrinkles or tears when selecting a location for sampling. Care should be taken to minimize distortion and compression of the specimen while cutting. Set up and calibrate the micro-CT instrument according to the manufacturer’s specifications. Place the specimen into an appropriate holder and support the specimen with a ring of low-density material. This will allow the central portion of the specimen to lay horizontal and be scanned without having any other materials directly adjacent to its upper and lower surfaces. Measurements should be taken in this region. The 3D image field of view is approximately 10 mm on each side in the xy-plane with a resolution of approximately 5600 by 5600 pixels, and with a sufficient number of 1.8 microns thick slices collected to fully include the z-direction of the specimen. The reconstructed 3D image contains isotropic voxels of 1.8 microns. Images were acquired with the source at 70 kVp and 57 µA with a 0.1 mm aluminum filter. A total of 3400 projections images were obtained with an integration time of 800 ms and 4 averages. The projection images are reconstructed into the 3D image and saved in 16-bit format to preserve the full detector output signal for analysis. For each µCT instrument, these current and voltage settings should be optimized to produce the maximum contrast in the projection data with sufficient x-ray penetration through the specimen, but once optimized held constant for all substantially similar specimens.

### 3) Image Processing Method

A square section of the nonwoven is cropped to approximately 3.2 mm × 3.2 mm from the µCT voxel data. The analysis is performed on the 8-bit dataset that completely intersects the minimum X border, the maximum X border, the minimum Y border and the maximum Y border. A small non-material buffer region will exist between the minimum Z border and the maximum Z border. This region will consist of air or packing material. A Material Threshold is determined for the specimen by Otsu’s method using Matlab’s built in “multithresh” function. This threshold should identify the nonwoven material while nearly eliminating noise and packing material. Stray packing material should be removed from the data manually when necessary.

Applying the Material Threshold results in a binary dataset with the fiber voxels labeled as “1”. The approximate thickness of the fibers can be accurately estimated with a Local Thickness Map (LTM). A LTM dataset is the same size as the fiber dataset. Every fiber voxel location in the LTM is assigned the radius value of the largest sphere within the fiber’s morphology that encloses that fiber voxel. Finding and using local thickness values was first discussed in:

Hildebrand T, Rüegsegger P (1997) A new method for the model-independent assessment of thickness in three-dimensional images. J. Microsc. 185: 67-75.

A LTM algorithm was implemented in Matlab and executed on the fiber labelled dataset. The bisecting profile of the fibers is nearly circular making the radius of the maximum enclosing sphere a good estimation of the radius of the fiber at that voxel location. A Radius Threshold which separates the smaller diameter fiber voxels from the larger diameter fiber voxels is determined for the dataset by passing all fiber voxels to Matlab’s “multithresh” function. LTM values greater than the Radius Threshold will result in a binary mask where nearly all the large diameter fiber voxels are labeled as “1”.

µCT scanning is an x-ray technology where intensity values are proportional to the density of the fiber material. A voxel is a cubic volume in space. The intensity of each fiber voxel therefore is proportional to the weight of the fiber within that cubic volume. Basis Weight is defined as weight over area. Therefore, summing all fiber voxel values and dividing that summation over the X-Y footprint (3.2 mm × 3.2 mm) will give a value that is proportional to the Basis Weight of the specimen. The large diameter fiber ratio of that Basis Weight can be defined according to the following formula:

$$\begin{matrix}\text{Large Fiber Ratio Of Basis Weight} = \\ \frac{\sum L a r g e F i b e r V o x e l I n t e n s i t i e s}{\sum A l l F i b e r V o x e l I n t e n s i t i e s}\end{matrix}$$

The small diameter fiber percentage of that basis weight can be defined as below and reported to the nearest 0.1%:

Small Fiber Of Basis Weight % = 100% × (1 – Large Fiber Ratio Of Basis Weight) Repeat the measurement and analysis on multiple specimens. The arithmetic mean of the “Small Fiber Of Basis Weight” from at least three replicate specimens will be reported as the weight ratio (%) of the garment facing layer to the nearest 0.1 %.

### Thickness Under Compression

### Sample Preparation

To obtain a sample from the crotch region (30) of the finished absorbent article, remove any cuffs and deactivate any elastic portion on both sides so that the remaining article can be placed flat on a bench. For a pants-type article, tear open the elastic belt along the side seam in advance. A square-shaped sample with the size of 110 mm × 110 mm is cut out from the front side of the article using a paper trimmer, which has one side centrally located along the lateral centerline of the article and extends towards the elastic belt. The sample needs to be pre-conditioned in a room maintained at 23 ± 2° C. and 50 ± 5 % relative humidity, for at least 4 hours prior to testing.

### Thickness Under Compression

Thickness Under Compression of the samples are measured using the Fabric Touch Tester (FTT® M293) and FTT® system software available from SDL Atlas, or equivalent. FTT® system includes five modules (i.e., compression, bending, surface friction, roughness, and thermal properties) that can be activated at the same time for recording the dynamic responses from the samples if needed. The measurement for Thickness Under Compression only requires the compression module. The instrument is calibrated according to the manufacturer’s instructions, using the standard calibration fabrics provided along with the instrument. All the testing is performed in a room maintained at 23 ± 2° C. and 50 ± 5 % relative humidity. The test procedures are conducted according to the operating instructions given in the FTT M293 manual.

The 110 mm × 110 mm sample with garment facing side upward is placed centrally on the lower plate in the FTT® system. The compression measurement is undertaken with single surface testing mode, when the sample is pushed downwards by the upper plate in the FTT® system that applies a continuously increasing normal force from 0 to 8470 gf (i.e. 0 to 70 gf/cm² in pressure).

Compression Work (CW) denotes the total work done on the sample during the compression process. Integral of the compression curve according to equation (1) is calculated to give the value of Compression Work in the unit of gf × mm, wherein Dₐ is the initial sample thickness at zero pressure, D_{c} is minimum sample thickness at maximum pressure, F is the measured force and D is the measured thickness during compression. The reported values will be the arithmetic mean of five replicate samples to the nearest 1 gf × mm.

$$C W = {\int }_{{D}_{u}}^{{D}_{c}} F d D$$

Thickness Under Compression of the sample is the measured thickness under the pressure of 41 gf/cm², during the compression test. The reported values will be the arithmetic mean of five replicate samples to the nearest 0.01 mm.

### MD Tensile Strength/Basis Weight

MD tensile strength of a specimen is measured according to NWSP 110.4-09 with conditions below.
Test Speed: 100 mm/min
Sample Width: 50 mm
Sample length: sufficiently longer than gauge length
Gauge Length: 100 mm

### Thickness/Basis Weight

The Compression Average Rigidity (CAR), Standard Thickness (T), and Bending Work (BW) values are measured on a nonwoven test sample using a Fabric Touch Tester M293 (FTT), available from SDL Atlas USA, Rock Hill, SC, interfaced with a computer running FTT system software. According to SDL Atlas, the FTT objectively and quantitatively characterizes skin touch comfort by measuring various mechanical and surface properties. The FTT instrument offers a variety of assessment modules to measure these properties. The FTT Test utilizes the Compression Module, which compresses a sample between two plates while recording the applied normal force and corresponding distance between the plates during a compression and recovery cycle. The FTT Test also utilizes the Bending Module, which bends a sample over a bending bar while recording the bending force and corresponding bending angle. The recorded data is analyzed by the FTT software to calculate the CAR, T, and BW values. The instrument operation and testing procedures are performed according the instrument manufacture’s specifications.

### 1 Sample Preparation

When a nonwoven is available in a raw material form, a rectangular test sample with a size of 310 mm × 90 mm is cut from the raw material. When a nonwoven is a component of a finished product, the nonwoven is removed from the finished product using a razor blade to excise the nonwoven from other components of the finished product to provide a nonwoven test sample with a size of 310 mm × 110 mm. A cryogenic spray (such as Cyto-Freeze, Control Company, Houston TX) may be used to remove the nonwoven specimen from other components of the finished product, if necessary. Equilibrate all samples at TAPPI standard temperature and relative humidity conditions (23° C. ± 2 C° and 50 % ± 2 %) for at least 4 hours prior to conducting the FTT testing, which is also conducted under TAPPI conditions.

### 2 Testing Procedure

The FTT instrument is calibrated according to the manufacturer’s instructions using the provided standard calibration fabric. The test sample is placed into the instrument according to the manufacturer’s instructions, with the appropriate amount of the sample laying on the compression plate and the remaining portion resting on the adjacent bending platform. The test sample should be laying flat and tension free prior to initiating the test. The compression and bending tests are initiated and performed according to the manufacturer’s instructions.

When testing is complete, the FTT software displays values for CAR, T, and BW. Record each of these values. The test piece is then removed from the instrument and discarded. This testing procedure is performed individually on the other four replicate test samples.

The arithmetic means of the five recorded test result values for CAR, T, and BW are calculated and reported. Report the individual average values of CAR to the nearest 1 gf/mm³, T to the nearest 0.01 mm, and BW to the nearest 1 gf•mm•rad.

### EXAMPLE

### Nonwoven Examples

A multi-fiber layer nonwoven of the present invention with tradename N_HB2008 available from Wisdom Nonwoven Co. LTD having Lot No. WSFS3C210712 was subjected to tests herein for obtaining the Fiber Diameter. Such multi-fiber layer nonwoven had 2 distinctive layers of fibers, and the fiber diameter of those layers were obtained according to measurements herein. The SEM photos of FIGS. 3A-3C were taken during the process of this measurement. Results are provided in Table 1A. Two types of such multi-fiber layer nonwoven, Nonwoven Example 1 having a basis weight of 20, and Nonwoven Example 2 having a basis weight of 25, were used in the Article Examples below. The weight ratio (%) of the garment facing layers were obtained according to measurements herein, and provided in Table 1B.

TABLE 1A

| Layer | Fiber Diameter (µm) |
|---|---|
| Garment facing | 10.4 |
| Wearer facing | 16.2 |

TABLE 1B

| Example | Weight ratio of garment-facing layer (%) |
|---|---|
| Nonwoven 1 | 51.0 |
| Nonwoven 2 | 40.3 |

### Article Examples

Article Examples 1-2 and Comparative Examples 1-3 were obtained as such and subject to the tests described below.

Example 1: Size 4 (L-size) belt-type pant article (Lot No. 20210828) having the Nonwoven Example 1 form the outer sheet and Nonwoven Example 2 form the outer cover layer, and having the configuration, elastic bonding, and pattern of discrete bond units of FIG. 6, elastic profile and other properties of Table 2 below, and including the intermediate layer in the central chassis.

Example 2: Size 4 (L-size) belt-type pant article (Lot No. 20210226) having the Nonwoven Example 2 form the outer cover layer, and having the configuration, elastic bonding, and pattern of discrete bond units of FIG. 6, and elastic profile and other properties of Table 2 below, and including the intermediate layer in the central chassis.

Comparative Example 1: “Ichiban Pants” Size 4 (Lot No. 20210223) purchased in February 2021 in PRC having the outer sheet and outer cover layer formed by a nonwoven similar to Nonwoven Example 1, however not having fibers with diameter less than 12 µm.

Comparative Example 2: “Huggies Penguin” Size 4 purchased in PRC having the outer sheet and outer cover layer formed by a nonwoven similar to Nonwoven Example 1, however not having fibers with diameter less than 12 µm. Lot No. 20210225 purchased in June 2021 was used for the measurement to obtain “Thickness Under Compression”, and Lot No. 20210412 purchased in July 2021 was used for the other tests.

Comparative Example 3: “Baby Care Royal Weak Acid” Size 4 purchased in PRC having the outer sheet and outer cover layer formed by a nonwoven similar to Nonwoven Example 1, however not having fibers with diameter less than 12 µm. . Lot No. 20200902 purchased in June 2021 was used for the measurement to obtain “Thickness Under Compression”, and Lot No. # 20210402 purchased in July 2021 was used for the other tests.

TABLE 2

| | |
|---|---|
| Zone | Examples 1-2 Dtex / elongation / Number of elastics |
| Front waist zone | 470Dtex / 180% / 7 |
| Front tummy zone | 940Dtex / 190% / 8 with tummy cut (*1) |
| Front leg zone | 470Dtex / 180% / 2 with tummy cut (*1) |
| Back waist zone | 470Dtex / 180% / 4 940Dtex/ 130% / 3 |
| Back tummy zone | 940Dtex/ 130% / 1 470Dtex / 180% /4 470Dtex / 240% / 1with tummy cut (*1) |
| Back leg zone | 470Dtex / 240% / 2 with tummy cut (*1) |

(*1) “Tummy cut” in Table 2 refers to deactivation of elasticity at the transverse central area of elastic strands resulting in 68% effective length of elasticity.

### 1. Technical Measurements

The Thickness Under Compression was measured according to methods herein and results provided in Table 3 below.

TABLE 3

| | Example 1 | Comparative Example 1 | Comparative Example 2 | Comparative Example 3 |
|---|---|---|---|---|
| Thickness Under Compression (mm) | 2.85 | 2.35 | 3.44 | 3.60 |

### 2. Consumer Acceptance Test 1

60 panelists who were caregivers of babies using Size 4 (L size) pant diapers and having a mixture of usage experience of major brands of similar price range used in the test were recruited. There were about equal number of caregivers of boy and girl babies and having a weight of 9-14 kg. Five (5) finished product test samples (including Example 1 and Comparative Examples 2-3) were provided to the panelists to touch and feel the center of the product with their hands one by one. Each respondent was asked to fill in a questionnaire individually after touching the test sample one by one. In the questionnaire, there were 4 values as found in Table 4, and each respondent was requested to sort and rate the test samples against those values using the ratings from 1 to 10, which were scored as such: ‘1= poor, 10 = excellent’. The scores were averaged .

TABLE 4

| Values | Example 1 | Comparative Example 2 | Comparative Example 3 |
|---|---|---|---|
| Overall rating | 75(*2) | 58 | 53 |
| Overall softness | 83(*3) | 76 | 60 |
| Belt softness | 83(*2) | 75 | 72 |
| Crotch softness | 84(*2) | 70(*3) | 55 |

(*2) These scores were statistically significantly better against Comparative Examples 2-3 at 90% confidence level.
(*3) These scores were statistically significantly better against Comparative Example 3 at 90% confidence level.

According to the test result in Table 4, Example 1 which meets the requirements of the present invention has statistically significantly higher values compared to the Comparative Examples.

### 2. Consumer Acceptance Test 2

107 panelists who were caregivers of babies using Size 4 (L size) pant diapers and having diaper usage of at least 3 pads/day in the past 7 day were recruited. There were about equal number of caregivers of boy and girl babies and having a weight of 9-14 kg.

Each panelist were provided enough test products of Example 1 and Comparative Example 1 for 4 consecutive days usage each. Panelists were asked to use only the test products, while following their normal usage frequency/habits. At the very beginning, the panelists were asked to take 1 pad to touch and feel, and asked to fill in the “Before usage” questions as in Table 5 by rating from 1 to 10, which were scored as such: ‘1= poor, 10 = excellent’. After the 4 day usage the panelists were asked to repeat the same. The scores were averaged and provided in Table 5.

TABLE 5

| Values | Example 2 | Comparative Example 1 |
|---|---|---|
| Before usage - Overall rating | 67(*4) | 59 |
| Before usage - Overall softness | 72(*4) | 58 |
| Before usage - Crotch softness | 72(*4) | 60 |
| After usage - Overall rating | 65 | 59 |
| After usage - Overall softness | 81(*4) | 63 |
| After usage - Crotch softness | 77(*4) | 66 |

(*4) Statistically significantly better against Comparative Example 1 at 90% confidence level.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.” Further, every numerical range given throughout this specification includes every narrower numerical range that falls within such broader numerical range.

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An absorbent article comprising a liquid permeable topsheet, a liquid impermeable backsheet, an absorbent core disposed between the topsheet and the backsheet, and an outer cover layer for covering the garment-facing side of the backsheet;
wherein the absorbent article has a Thickness Under Compression of from about 2.7 mm to about 4.0 mm; and
wherein the outer cover layer is formed by a muti-fiber layer nonwoven having a basis weight of from about 16gsm to about 35gsm and comprising a garment facing layer and a wearer facing layer, the garment facing layer comprising fibers having a diameter from about 7 µm to about 11 µm, and the wearer facing layer comprising fibers having a diameter from about 13 µm to about 24 µm, and wherein a weight ratio of the garment facing layer is from about 20% to about 70% of the multi-fiber layer nonwoven.

2. The absorbent article of claim 1, wherein the fibers of the multi-fiber layer nonwoven are air-through carded fibers, and wherein the layers of the multi-fiber layer nonwoven are air through bonded.

3. The absorbent article of claim 1, wherein the absorbent core comprises a high loft material encompassing superabsorbent polymer particles.

4. The absorbent article of claim 1, wherein the absorbent core comprises an absorbent layer having superabsorbent polymers disposed between first and second layers of nonwoven material immobilized by a fibrous layer of thermoplastic adhesive material.

5. The absorbent article of claim 4, comprising an acquisition system between the topsheet and the absorbent core, wherein the acquisition system comprises viscose.

6. The absorbent article of claim 4, comprising an intermediate layer disposed between the absorbent core and the backsheet, wherein the intermediate layer has an MD Tensile/Basis Weight of no greater than about 0.75 N/5 cm/g/m², and a Thickness/Basis Weight ratio of no less than about 0.078 mm/g/m².

7. The absorbent article of claim 6, wherein the intermediate layer comprises a first thermoplastic fiber having a first melting temperature and a second thermoplastic fiber having a second melting temperature, and wherein a difference between the first melting temperature and the second melting temperature is at least about 40° C.

8. The absorbent article of claim 7, wherein the first thermoplastic fibers are heat-fused to one another, and wherein the second thermoplastic fibers are hollow fibers or shaped fibers which are not heat-fused to one another.

9. The absorbent article of claim 6, wherein the intermediate layer comprises a nonwoven web selected from the group consisting of an air-through bonded nonwoven comprising staple fibers, carded calendar bonded nonwovens comprising staple fibers and combinations thereof, a spunlace nonwoven comprising staple fibers, an air-through bonded nonwoven comprising spunlaid fibers and a spunlace nonwoven comprising spunlaid fibers.

10. The absorbent article of claim 1, comprising an application means, wherein the application means is selected from the group of a fastening means and an elastic belt.

11. The absorbent article of claim 10, wherein the application means is the elastic belt extending in the transverse direction, wherein the elastic belt comprises a laminate comprising an inner sheet, an outer sheet, and a plurality of elastic members running in the transverse direction;
wherein the muti-fiber layer nonwoven forming the backsheet nonwoven also forms the outer sheet.

12. The absorbent article of claim 11, wherein the inner sheet has a melting point of no more than about 165° C.

13. An absorbent article comprising a liquid permeable topsheet, a liquid impermeable backsheet, an absorbent core disposed between the topsheet and the backsheet, and an outer cover layer for covering the garment-facing side of the backsheet;
wherein the absorbent article has a Thickness Under Compression of from about 2.7 mm to about 4.0 mm;
wherein the outer cover layer is formed by a muti-fiber layer nonwoven having a basis weight of from about 16gsm to about 35gsm and comprising a garment facing layer and a wearer facing layer, the garment facing layer comprising fibers having a diameter from about 7 µm to about 11 µm, and the wearer facing layer comprising fibers having a diameter from about 13 µm to about 24 µm, and wherein a weight ratio of the garment facing layer is from about 20% to about 70% of the multi-fiber layer nonwoven; and
wherein the fibers of the multi-fiber layer nonwoven are air-through carded fibers, and wherein the layers of the multi-fiber layer nonwoven are air through bonded.

14. The absorbent article of claim 13, wherein the absorbent core comprises a high loft material encompassing superabsorbent polymer particles.

15. The absorbent article of claim 13, wherein the absorbent core comprises an absorbent layer having superabsorbent polymers disposed between first and second layers of nonwoven material immobilized by a fibrous layer of thermoplastic adhesive material.

16. The absorbent article of claim 14, comprising an acquisition system between the topsheet and the absorbent core.

17. The absorbent article of claim 14, comprising an intermediate layer disposed between the absorbent core and the backsheet, wherein the intermediate layer has an MD Tensile/Basis Weight of no greater than about 0.75 N/5cm/g/m², and a Thickness/Basis Weight ratio of no less than about 0.078 mm/ g/m².

18. The absorbent article of claim 17, wherein the intermediate layer comprises a first thermoplastic fiber having a first melting temperature and a second thermoplastic fiber having a second melting temperature, and wherein a difference between the first melting temperature and the second melting temperature is at least about 40° C.

19. The absorbent article of claim 18, wherein the first thermoplastic fibers are heat-fused to one another, and wherein the second thermoplastic fibers are hollow fibers or shaped fibers which are not heat-fused to one another.

20. An absorbent article comprising a liquid permeable topsheet, a liquid impermeable backsheet, an absorbent core disposed between the topsheet and the backsheet, and an outer cover layer for covering the garment-facing side of the backsheet;
wherein the absorbent article has a Thickness Under Compression of from about 2.7 mm to about 4.0 mm; and
wherein the outer cover layer is formed by a muti-fiber layer nonwoven having a basis weight of from about 16gsm to about 35gsm and comprising a garment facing layer and a wearer facing layer, the garment facing layer comprising fibers having a diameter of about 11 µm or less, and the wearer facing layer comprising fibers having a diameter from about 13 µm or more, and wherein a weight ratio of the garment facing layer is from about 20% to about 70% of the multi-fiber layer nonwoven.
